(19) United States
(12) Patent Application Publication
(10) Pub. No.: US 2023/0139724 A1
(43) Pub. Date: May 4, 2023

(54) SYSTEM AND METHOD FOR TESTING EFFECTS OF CHEMICAL COMPOUNDS ON COGNITIVE FUNCTION

(71) Applicant: CCLabs Pty Ltd, Melbourne (AU)

(72) Inventors: Hon Weng Chong, Melbourne (AU); Brett Joseph Kagan, Melbourne (AU); Andy C. Kitchen, Melbourne (AU)

(21) Appl. No.: 17/515,269

(22) Filed: Oct. 29, 2021

(51) Int. Cl. G01N 33/50; C12N 13/00; B01L 3/00

(52) U.S. Cl. CPC G01N 33/5058; C12N 13/00; G01N 33/502; B01L 3/502; B01L 2200/025; B01L 2200/10; B01L 2200/16; B01L 2300/0636; B01L 2300/0645; B01L 2300/0819

(57) ABSTRACT

A method of performing in vitro testing of an effect of a chemical compound on biological neurons is disclosed in embodiments. The method includes training in vitro biological neurons to perform a task representative of a cognitive function of the neurons; determining a first cognitive function value of the neurons based on an ability of the neurons to perform the task; exposing the neurons to a chemical compound; determining a second cognitive function value of the neurons based on the ability of the neurons to perform the task during and/or after exposure to the chemical compound; comparing the second cognitive function value to the first cognitive function value; and determining an effect of the chemical compound on the cognitive function of the neurons based on a difference between the second cognitive function value and the first cognitive function value.

### TECHNICAL FIELD

Embodiments of the present disclosure relate, in general, to testing of chemical compounds, and in particular to use of a neurological computation and experimentation platform for drug testing, such as for testing the effects of pharmaceuticals on cognitive function.

### BACKGROUND

All new drugs (also referred to herein as pharmaceuticals) must be evaluated before they can be sold. Drugs are tested to ensure that they work correctly and that their health benefits outweigh their risks. Only once independent and unbiased review of a drug establishes that the drug's health benefits outweigh its risks is a drug approved for sale.

Presently there are many different types of tests that are performed on drugs to assess both the effectiveness of drugs and any risks associated with the drugs. Unfortunately, the only known technique for testing the effect of a drug on cognitive function is in vivo testing, in which the drug is actually used on an individual and the effects of the drug on that individual's cognitive function are measured. Such testing of drugs on people (or animals) is expensive and time consuming, and poses risks to those people (or animals) on which the drug is tested. Additionally, testing on non-human animals is widely known to have limited applicability to humans and cannot account for specific human genetic differences.

### BRIEF DESCRIPTION OF THE DRAWINGS

The embodiments described herein will be understood more fully from the detailed description given below and from the accompanying drawings, which, however, should not be taken to limit the application to the specific embodiments, but are for explanation and understanding only.

FIG. 1A illustrates an example system architecture for a biological computing platform, in accordance with one embodiment.

FIG. 1B illustrates an example in vitro chemical compound testing system, in accordance with one embodiment.

FIG. 2 is a sequence diagram illustrating one embodiment for a method of using a biological computing platform.

FIG. 3 is a sequence diagram illustrating one embodiment for a method of providing reinforcement learning to biological neurons in a biological computing platform.

FIG. 4 is a flow diagram illustrating one embodiment for a method of using a biological computing platform.

FIG. 5 is a flow diagram illustrating one embodiment for a method of testing the effect of a chemical compound on cognitive function of neurons.

FIG. 6 is a flow diagram illustrating one embodiment for a method of testing the effect of a chemical compound on cognitive function on many different samples of neurons.

FIG. 7 illustrates an example computing device, in accordance with one embodiment.

### DETAILED DESCRIPTION OF THE PRESENT DISCLOSURE

Described herein are embodiments of an in vitro chemical compound testing system usable to test the effects of chemical compounds such as pharmaceuticals on cognitive function of neurons. Also described herein are methods of performing in vitro testing of the effects of chemical compounds on cognitive function of neurons. Embodiments may be practiced for drug discovery, drug efficacy testing, drug side effect testing, and so on. Testing may be performed using generic cell lines and/or using neurons grown from a stem cell line from a particular patient of interest, for example.

In embodiments, a biological computing platform, which may be implemented as a biological computing research platform (also referred to as a neural computation and experimentation platform) and/or an in vitro chemical compound testing system can be used to perform in vitro chemical compound testing. The biological computing platform may be a biological computing cloud platform that provides network access to biological neural networks (e.g., exposes biological neural network resources through the cloud). In one embodiment, the biological computing platform externalizes networks of biological neurons (e.g., cortical neurons) and provides an interface between the biological neural network and a virtual environment executed on a computing device. Accordingly, the biological computing platform creates an afferent (e.g., vision or other input)/efferent (e.g., motor or other output) loop between the biological neural network and the virtual environment.

The biological computing platform may be used to train groups or clusters of neurons to perform a task (e.g., to interact with a virtual environment such as play a computer game). The task may be an interactive task that requires problem solving and/or decision making. The degree of success of the trained neurons to perform the task may be representative of a cognitive function of the neurons (e.g., may be used as a proxy for cognitive function of the neurons). Once the biological computing platform has been used to train a group or cluster of neurons to perform a task, a cognitive function value may be determined based on the ability of the neurons to perform the task. For example, if the task is to play the computer game Pong, then the cognitive function value may be an amount of time that the neurons are able to play the game before the game ends and/or a level of the game that can be reached by the neurons. A baseline cognitive function value may be determined while the neurons are not under the effects of any chemical compound. The neurons may be exposed to a chemical compound (or multiple chemical compounds), and another cognitive function value may be determined during or after exposure to the chemical compound(s) (e.g., while the neurons are under the effect of the chemical compound). The effect of the chemical compound(s) on the cognitive function of the neurons may then be determined based on the difference between the post-exposure cognitive function value and the baseline cognitive function value. Such tests may be performed for many different groups or clusters of neurons, which may be from generic cell lines and/or from stem cell lines associated with specific genotypes. Statistical analysis may be performed on the results to determine the effects of the chemical compound over a large population.

In some embodiments, a determined cognitive function value is, or is based at least in part on, a neuro criticality value. Neuro criticality of the neurons may be measured, and a baseline neuro criticality value may be determined for the neurons while they are not under the influence of any chemical compound. The neurons may then be exposed to one or more chemical compound, and neuro criticality may again be measured. The neuro criticality values of the neurons under the influence of the chemical compound may be compared to the baseline neuro criticality value of the neurons, and a difference between the chemical compound-influenced neuro criticality value(s) and the baseline neuro criticality value(s) may be used to determine the effects of the chemical compound on the neurons.

The mechanisms that have been developed to encode and decode artificial neural networks are generally inapplicable to biological neural networks. In an artificial neural network, data is frequently encoded as floating point vectors, which is then input into the artificial neural network. The artificial neural networks then generally are trained to output further vectors, which are easily decodable. However, in a biological neural network, data is encoded as spikes of action potentials (e.g., voltage variation across a cell membrane) of a population of biological neurons. In embodiments, the biological computing platform acts as an encoder/decoder to generate signals that can be interpreted by biological neural networks and to decode output signals generated by the biological neural networks.

Embodiments provide a biological computing platform that may include a multielectrode array (MEA) and/or an optics-based equivalent to an MEA that uses optical input and/or output signals connected to a computing device. The computing device may be a physical computing device or a virtual computing device. The computing device may execute an interface (referred to herein as an MEA interface though it can also interface with other systems such as a substrate comprising an optics-based or optical system) that enables the computing device to communicate with the MEA and/or other system (and with a biological neural network contained within the MEA and/or other system). The optical system may be referred to as an optical MEA, a phosomilia system, or an optical energy interchange system. The computing device may additionally execute an experiment logic or virtual environment that interfaces with the MEA interface. The MEA interface may receive digital input signals from the experiment logic or virtual environment, convert the digital input signals into instructions for the MEA and/or other system, and then send the instructions to the MEA and/or other system. The instructions may cause the MEA and/or other system to apply a plurality of electrical or optical impulses at excitation sites having coordinates on a 2D grid or other array of sites (e.g., excitation sites) in the MEA and/or other system. The MEA interface may additionally receive representations of electrical and/or optical signals measured at locations on the 2D grid or other array from the MEA and/or other system, generate responses for the experiment logic or virtual environment based on the representation, and send the responses to the experiment logic or virtual environment. In this manner, the MEA interface enables the virtual environment or experiment logic to interact with the biological neural network on the MEA and/or other system.

In one embodiment, a biological computing platform includes an MEA connected to a computing device. The MEA may include a two-dimensional (2D) grid of excitation sites, a plurality of biological neurons disposed on the MEA, and a processing device or integrated circuit. Alternatively, the MEA may be a circuitless chip, which may be connected to a processing device or integrated circuit (e.g., via a printed circuit board). The processing device may be a complementary metal-oxide-semiconductor (CMOS) chip. In one embodiment, the processing device is a component of a system on a chip (SoC) that includes a network adapter, an analog to digital converter and/or a digital to analog converter.

The computing device may receive or generate a digital input signal, convert the digital input signal into instructions for the plurality of electrical or optical impulses, and send the instructions to the MEA and/or other system. The MEA and/or other system may use a digital to analog converter (DAC) to convert the instructions from a digital form into an analog form, and the processing device of the MEA and/or other system may apply the plurality of electrical or optical impulses at excitation sites having coordinates on the 2D grid or other array of excitation sites. In embodiments, optical stimulation designed to elicit an electrical response in cells and electrical stimulation to elicit an electrical response in cells are both referred to as electrical signals. One or more sensors and/or the processing device may measure electrical signals output by one or more of the plurality of biological neurons at coordinates of the 2D grid or other array. In embodiments, excitations of neurons may be captured using optical sensors. For example, when neurons fire such firing may be detected optically by one or more optical sensors. Thus, the impulses output by neurons discussed herein may be captured as optical signals that represent an electrical state of the neurons. Accordingly, any discussion of electrical signals output by neurons herein may be measured as optical signals as detected by one or more optical sensors. The processing device may then generate a representation of the electrical signals and/or optical signals, and may send the representation back to the computing device. The computing device may convert the representation into a response readable by a virtual environment or experiment logic, and may send the response to the experiment logic or virtual environment.

In some embodiments, the biological computing platform is a fully optical system that lacks an MEA. Alternatively, the biological computing platform may include an MEA with an optical system that provides optical signals to neurons and/or that receives optical signals from the neurons. It should be understood that embodiments discussed herein with reference to an MEA also apply to alternatives in which a fully optical interface is used rather than an MEA as well as hybrid systems that include an MEA and optical components (e.g., image sensors and/or light sources). The optical interface may perform a similar function as that traditionally performed by an MEA in such embodiments. Accordingly, references to an MEA also apply to optical components that perform a similar function as an MEA. Moreover, any electrical signals discussed herein may be modified such that optical signals are used instead of or in addition to electrical signals, including electrical signals delivered to neurons and electrical signals received from neurons.

In embodiments, the biological computing platform provides an interface between biological neurons and a virtual environment and/or experiment logic that can train the neurons to perform one or more task. The ability of the neurons to perform the task may be representative of the cognitive function of the neurons, and can be measured. Accordingly, the biological computing platform can be used to perform in vitro testing of cognitive function of biological neurons. In contrast, the only way to test cognitive function has historically been through in vivo testing. By enabling in vitro testing of cognitive function, embodiments provide an entirely new mechanism that can be used for drug testing. Using embodiments described herein, pharmaceutical companies can test the effects of drugs under development on the general population and/or on specific genotypes. Additionally, pharmaceutical companies can perform testing of multiple combinations of drugs to determine if there are any drug interactions that affect cognitive function. Such testing can be performed at scale by setting up many (e.g., hundreds or thousands) of samples of neurons, each in a separate MEA and each trained to perform a task, where performance of that task can be measured and used to assess cognitive function.

In one embodiment, a method of performing in vitro testing of cognitive function comprises training a first plurality of in vitro biological neurons to perform a task, wherein an ability of the first plurality of biological neurons to perform the task is representative of a cognitive function of the first plurality of in vitro biological neurons. The method further includes determining a first cognitive function value of the first plurality of in vitro biological neurons based on an ability of the first plurality of in vitro biological neurons to perform the task. The method further includes exposing the first plurality of in vitro biological neurons to a chemical compound. The method further includes determining a second cognitive function value of the first plurality of in vitro biological neurons based on the ability of the first plurality of in vitro biological neurons to perform the task during or after exposure to the chemical compound. The method further includes comparing the second cognitive function value to the first cognitive function value. The method further includes determining an effect of the chemical compound on the cognitive function of the first plurality of in vitro biological neurons based on a difference between the second cognitive function value and the first cognitive function value. The method may be performed multiple times for the first plurality of biological neurons, and may also be performed one or more times for other sets of biological neurons of the same and/or different genotypes. Statistical analysis may be performed on the results to determine an effect of the chemical compound on cognitive function of neurons.

The determined cognitive function is more than a simple observation of electrical activity. Electrical activity is a physiological measure that does not correlate to cognitive function. In contrast, embodiments provide a measurable metric for cognitive function. Values for this metric can be determined before and after exposure to chemical compounds, and differences between these values can be determined and used to assess a quantifiable effect of the chemical compound on cognitive function.

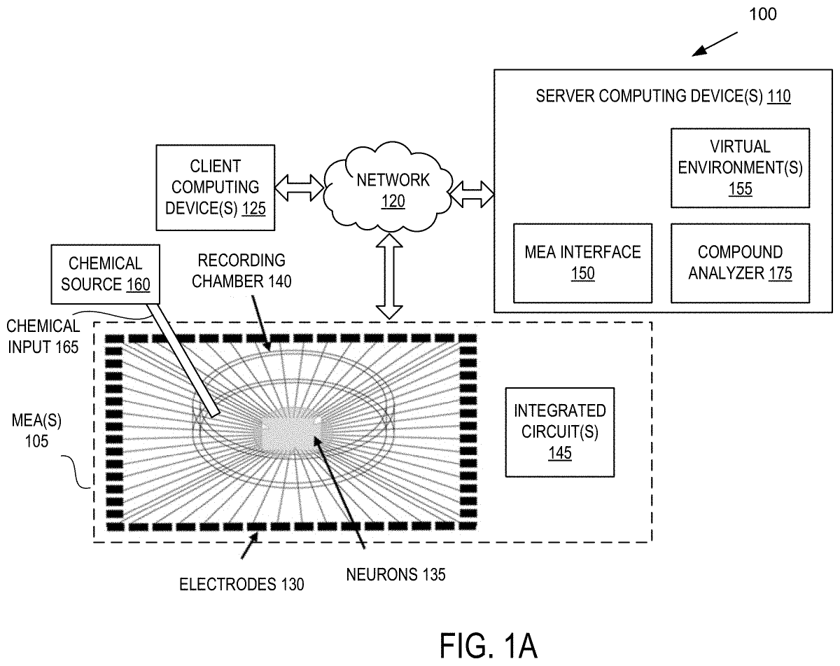

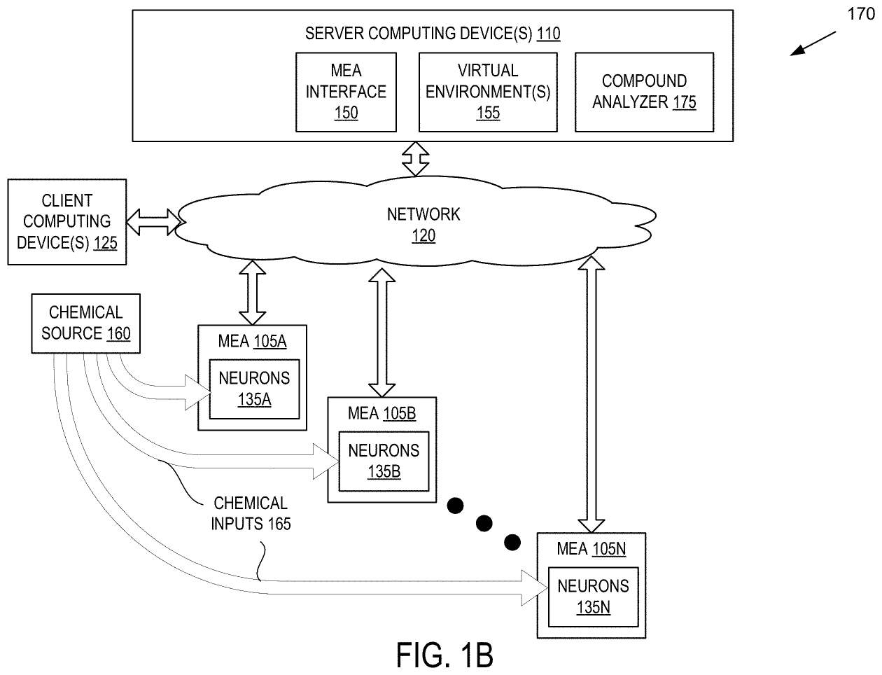

FIG. 1A illustrates an example system architecture for a biological computing platform 100, which may be used to test a cognitive function of neurons in accordance with one embodiment. As shown, the biological computing platform 100 includes one or more MEA 105 connected to one or more server computing devices 110 via a network 120. The network 120 may be a local area network, a wide area network, a private network (e.g., an intranet), a public network (e.g., the Internet), or a combination thereof. The connection between the MEA(s) 105 and the server computing device(s) 110 may include wired connections, wireless connections, or a combination thereof. Alternatively, the MEA(s) 105 may be directly connected to server computing devices(s) 110 (e.g., via a wired or wireless connection).

The server computing devices 110 may include physical machines and/or virtual machines hosted by physical machines. The physical machines may be rackmount servers, desktop computers, or other computing devices. In one embodiment, the server computing devices 110 include virtual machines managed and provided by a cloud provider system. Each virtual machine offered by a cloud service provider may be hosted on a physical machine configured as part of a cloud. Such physical machines are often located in a data center. The cloud provider system and cloud may be provided as an infrastructure as a service (IaaS) layer. One example of such a cloud is Amazon's® Elastic Compute Cloud (EC2®).

The server computing devices 110 may host an MEA interface 150 and one or more virtual environments 155. The MEA interface 150 and virtual environment(s) 155 may be hosted on the same server computing device 110, or may be hosted on separate server computing devices, which may be connected via the network 120.

An MEA 105 (also known as a microelectrode array) is a device that contains multiple plates or shanks through which neural signals are obtained and/or delivered. The plates or shanks are generally arranged in a grid or other array, and serve as neural interfaces that connect neurons 135 to electronic circuitry. The MEA 105 includes a recording chamber 140 that houses many biological neurons 135 and/or a solution or other medium (e.g., a nutrient rich solution). These biological neurons 135 may be cultured neurons (e.g., cultured from stem cells or from a rat brain). The biological neurons 135 may be from a generic cell line, or may be from a cell line with specific traits to be tested. For example, the biological neurons 135 may be cultured from stem cells of a person having a particular genotype, or from a particular person for whom a test is to be performed, or from a person having a particular pathology.

Biological neurons create ion currents through their membranes when excited, causing a change in voltage between the inside and the outside of the cell. When recording, the electrodes on an MEA transduce the change in voltage from the environment carried by ions into currents carried by electrons (electronic currents). When stimulating, electrodes may transduce electronic currents into ionic currents through the media. This triggers the voltage-gated ion channels on the membranes of the excitable neurons, causing the neuron to depolarize and trigger an action potential.

The size and shape of a recorded signal may depend upon the nature of the medium (e.g., solution) in which the neuron or neurons are located (e.g. the medium's electrical conductivity, capacitance, and homogeneity), the nature of contact between the neurons and the electrodes (e.g. area of contact and tightness), the nature of the electrodes (e.g. its geometry, impedance, and noise), the analog signal processing (e.g. the system's gain, bandwidth, and behavior outside of cutoff frequencies), and data sampling properties (e.g. sampling rate and digital signal processing). For the recording of a single neuron that partially covers a planar electrode, the voltage at the contact pad is approximately equal to the voltage of the overlapping region of the neuron and electrode multiplied by the ratio the surface area of the overlapping region to the area of the entire electrode. An alternative means of predicting neuron-electrode behavior is by modeling the system using a geometry-based finite element analysis in an attempt to circumvent the limitations of oversimplifying the system in a lumped circuit element diagram.

The MEA(s) 105 can be used to perform electrophysiological experiments on dissociated cell cultures (e.g., cultures of biological neurons). With dissociated neuronal cultures, the neurons spontaneously form biological neural networks. The MEA(s) 105 may include an array of electrodes 130 and the recording chamber 140 that contains a living culture of biological neurons 135 in a nutrient rich solution or other solution that will keep the biological neurons alive. The array of electrodes 130 may be a planar array (e.g., a two-dimensional (2D) grid) or a three-dimensional (3D) array (e.g., a 3D matrix). The array of electrodes 130 that may be used to take measurements at 2D coordinates (or 3D coordinates) at high spatial and temporal resolution at excellent signal quality. Additionally, the array of electrodes 130 may be used to apply electrical impulses at the 2D coordinates or 3D coordinates.

One or more of the MEA(s) 105 may be an active MEA that includes an integrated circuit 145 (or multiple integrated circuits), such as a CMOS circuit. The integrated circuit(s) 145 may include processing logic (e.g., a general purpose or special purpose processor), a network adapter, a digital to analog converter (DAC), an analog to digital converter (ADC), and/or other components. The network adapter may be a wired network adapter (e.g., an Ethernet network adapter) or a wireless network adapter (e.g., a Wi-Fi network adapter), and may enable the MEA(s) 105 to connect to network 120. In one embodiment, the integrated circuit 145 includes a processing device, which may be a general purpose processor, a microcontroller, a digital signal processor (DSP), a programmable logic controller (PLC), a microprocessor or programmable logic device such as a field programmable gate array (FPGA) or a complex programmable logic device (CPLD). In one embodiment, the integrated circuit 145 includes a memory, which may be a non-volatile memory (e.g., RAM) and/or a volatile memory (e.g., ROM, Flash, etc.). In one embodiment, the integrated circuit 145 is a system on a chip (SoC) that includes the processing device, memory, network adapter, DAC, and/or ADC.

In one embodiment, one or more of the MEA(s) 105 is a passive MEA that is connected to one or more integrated circuits 145 via one or more leads and/or a printed circuit board (PCB).

In one embodiment, one or more of the MEAS 105 further includes an optical source that is capable of providing optical impulses to specified 2D coordinates in the 2D grid. The optical source may include light emitting elements (e.g., light emitting diodes (LEDs), light bulbs, lasers, etc.) that are capable of emitting light having one or more specified wavelengths. Accordingly, optogenics may be used to manipulate neural activity. Additionally, lasers of specific wavelengths may be used for highly accurate targeting of specific neurons. The response to optical stimulation may then be measured by the electrodes in the MEA(s) 105 or by image sensors, as described in greater detail below. Unlike electrical stimulation, light stimulation manipulates specific cells (e.g., neurons) that may express a targeted opsin protein, thereby making it possible to investigate the role of a subpopulation of neurons in a neural circuit. In some embodiments, immunofluorescence of specifically modified calcium that get cleaved and activated when they enter the neurons can also be paired with a camera to image activation of neurons.

In one embodiment, one or more of the MEAs 105 provide electrical stimulation to specified 2D coordinates in the 2D grid, but optical signals are measured. MEAS 105 may include one or more optical/image sensors capable of optically detecting electrical excitation of neurons and generating optical signals based on such detected electrical excitation of the neurons. Accordingly, optogenics may be used to detect neural activity. The optical sensors may include charge coupled devices (CCDs), complementary metal oxide (CMOS) devices, and/or other types of optical sensors.

Mechanisms for optically detecting neural activity are discussed in greater detail below. In some embodiments, immunofluorescence of specifically modified calcium that get cleaved and activated when they enter the neurons can be paired with one or more image sensors to image activation of neurons. In some embodiments genetically encoded voltage detectors may be introduced into cells at a given point and used to detect activation of neurons when stimulated with light. In some embodiments luciferase based reactions may be introduced into the cells and paired with another method of detecting voltage changes in neurons to detect changes in voltage without the need for external light stimulation.

In one embodiment, a fully optical system may be used instead of an MEA. In such an embodiment, a substrate on which the neurons are plated and/or additional components may include an optical source that is capable of providing optical impulses to specified 2D coordinates in a 2D grid. The optical source may include light emitting elements (e.g., light emitting diodes (LEDs), light bulbs, lasers, etc.) that are capable of emitting light having one or more specified wavelengths. Additionally, lasers of specific wavelengths may be used for highly accurate targeting of specific neurons. Additionally, the substrate and/or other components may include one or more optical sensors capable of optically detecting electrical excitation of neurons and generating optical signals based on such detected electrical excitation of the neurons. Accordingly, optogenics may be used to manipulate and detect neural activity.

In the case of an active MEA 105, on-chip signal multiplexing may be used to provide a large number of electrodes to achieve a high spatio-temporal resolution in recording of electrical signals and providing of electrical impulses. Moreover, weak neuronal signals can be conditioned right at the electrodes by dedicated circuitry units, which provide a large signal-to-noise ratio. Finally, analog-to-digital conversion may performed on chip, so that stable, digital signals are generated.

The MEA interface 150 may be responsible for translating between inputs/outputs of the virtual environment(s) 155 and the inputs/outputs of the MEA(s) 105. Each virtual environment 155 may include some processing logic that may receive inputs from one or more MEAs 105 and/or an external source, and that may generate outputs. One example of processing logic of a virtual environment is a machine learning model such as an artificial neural network, deep neural network, convolutional neural network, recurrent neural network, etc. Other machine learning models included in virtual environments may apply a k-nearest neighbors algorithm, a learning vector quantization, a self-organizing map, regression analysis, a regularization algorithm, and so on. Another example of processing logic of a virtual environment is an application executing on the server computing device. For example, the application may be a game (e.g., Pong), and the biological neurons 135 on the MEA 105 may be trained to play the game. The application may also be any other program that includes one or more tasks to be performed or problems to be solved, and the biological neurons 135 on the MEA 105 may be trained to perform the task or tasks.

The server computing device 110 may provide one or more application programming interfaces (APIs) that enable third parties to upload virtual environments 155 and connect those virtual environments 155 to one or more MEAs 105. Each virtual environment 155 may be assigned one or more MEAs 105, and may train the neurons 135 on those MEAs 105 to perform some task, as discussed in greater detail below. Each virtual environment 155 may be assigned a virtual environment identifier (ID), and each MEA 105 may be assigned an MEA ID. Virtual environment IDs may be associated with MEA IDs 105 in a database or other data store, which may be maintained by the server computing device(s) 110.

Once a virtual environment 155 is paired with an MEA 105, that virtual environment may begin providing digital input signals for the MEA 105. The virtual environment 155 may generate a digital input signal, which may be, for example, a vector (e.g., a sparse vector and/or floating point vector), a message complying with some communication protocol, or a 2D or 3D matrix of values. The MEA interface 150 may include information on the array of electrodes 130 of the MEA 105. This may include information on the number of electrodes 130 and how the electrodes 130 are arranged in the recording chamber 140 (e.g., for a 2D grid of electrodes, the number or rows and columns of electrodes). The MEA interface 150 may convert the digital input signal from the virtual environment 155 into instructions for one or more electrical or optical impulses, where each electrical or optical impulse instruction is associated with a 2D coordinate or a 3D coordinate. Each electrical or optical impulse instruction may further include information on an amplitude or intensity of the impulse to apply, a frequency or wavelength of the impulse to apply and/or a current of the impulse to apply. Accordingly, the information for each impulse may be a tuple that includes one or more of (x coordinate, y coordinate, z coordinate, intensity/amplitude, frequency/wavelength, current).

Once the MEA interface 150 converts the digital input signal from the virtual environment into information for one or more optical or electrical impulses, it sends the information to the appropriate MEA 105. An integrated circuit 145 of the MEA 105 then converts the information into one or more analog signals for the optical or electrical impulses (e.g., using a DAC), and applies the one or more analog signals to appropriate electrodes 130 (or light emitting elements) to apply the optical or electrical impulses at the specified coordinates and/or with the specified intensity/amplitude, frequency/wavelength, and so on.

In response to the application of the electrical or optical impulses at the specified coordinates, one or more of the biological neurons 135 in the biological neural network in the recording chamber 140 will generate an electrical signal. The electrodes 130 may be used as sensors to measure electrical signals that may occur at various coordinates within the array (e.g., the 2D or 3D grid of electrodes 130). For example, the integrated circuit 145 (e.g., a CMOS chip) may read electrical impulses received at the electrodes 130. Alternatively, separate sensors may be arranged in the recording chamber 140. The electrical signals output by the neurons 135 may be measured, and their coordinates may be associated with the measurements. Other information such as amplitude (e.g., voltage), current and/or frequency may also be measured. The integrated circuit 145 may then generate a digital representation of the one or more measured electrical signals (e.g., using a ADC). This digital representation may then be sent from the MEA 105 to the MEA interface 150.

When one or more biological neurons 135 in the biological neural network generate an electrical signal, in some circumstances this may cause one or more nearby biological neurons to also generate an electrical signal. The electrical signals of the one or more nearby biological neurons may or may not trigger still further biological neurons to also generate an electrical signal, which may trigger activity of still more neurons, and so on. Experimental recordings from groups of neurons have shown bursts of activity, so-called neuronal avalanches, with sizes that follow a power law distribution. In neuroscience, the critical brain hypothesis states that certain biological neural networks work near phase transitions. According to this hypothesis, the activity of the brain (or biological neural networks generally) transitions between two phases, one in which activity will rapidly reduce and die, and another where activity will build up and amplify over time. In neuro criticality, the biological neural network capacity for information is enhanced such that subcritical, critical and slightly supercritical branching processes may describe how biological neural networks function. Neuro criticality (which may have a target neuro criticality value) refers to the value or point of the phase transition. The point of the phase transition is the amount of activity that is at a tipping point, below which damping forces prevail (and neural activity quickly dies out), and above which reinforcement forces prevail (and there is an exponential explosion of activity). Neuro criticality implies that on average each time a neuron fires (e.g., generates an electrical signal), this causes about one other neuron to also fire. However, some inputs (that are above the target neuro criticality value) can cause cascades of activity while other inputs (that are below the target neuro criticality value) can cause very little activity.

In embodiments, one or more neuro criticality values of a biological neural network are measured. These measured neuro criticality values may then be used to enhance, predict, and/or achieve computation on a device. For example, statistical markers for neuro criticality in a biological neural network may be determined by analyzing electrical activity of the biological neural network. For example, electrical activity information may be input into processing logic that performs statistical analysis on the electrical activity information to identify cascades of electrical activity, determine distributions of electrical activity, determine how long the cascades last, determine paths formed by chains of firing neurons, and so on. Such information may be used to determine a neuro criticality value of a biological neural network. In embodiments, there may be a target neuro criticality value for a biological neural network. If a measured neuro criticality value is below a target criticality value, then the biological neural network may be determined to be below criticality. If the measured criticality value is above the target neuro criticality value, then the biological neural network may be determined to be above criticality. Being either above or below criticality can impair the functioning of the biological neural network. Accordingly, an ability to measure the criticality value of the biological neural network and determine whether it is at, above, or below criticality (e.g., a target neuro criticality value) can be useful in assessing cognitive function of the biological neural network.

In embodiments, one or more other measures of neural activity may also be measured and used to enhance, predict and/or achieve computation on a device. Such other measures may measure information content, complexity, entropy, or a combination thereof. Any such measures may be used separately or together with neuro criticality in embodiments.

In one embodiment, one or more cameras are used to measure activated neurons. Specifically modified calcium may be used to cause the neurons to fluoresce under particular circumstances. The calcium may be cleaved and activated when it enters a neuron (which may happen when a neuron or pair of neurons is activated). The cleaving of the calcium may cause it to exhibit immunofluorescence. The one or more cameras can detect the fluorescence and determine a location that the fluorescence occurred. Alternatively, the MEA or computing device can receive the image from the camera and determine where the fluorescence occurred. In particular, the MEA or computing device may determine coordinates of where light was measured from the image. The MEA or computing device may then generate a digital representation of the locations at which light was detected (e.g., locations that exhibited immunofluorescence).

The MEA interface 150 may then generate a digital response message based on the digital representation received from the MEA 105. Generating the response message may include converting the representation into a format that is readable by the virtual environment 155. This may include converting the representation (e.g., which may be in the form of a matrix of values representing electrical and/or optical signals at various coordinates) into a sparse vector or tensor in one embodiment. The MEA interface 150 may then send the response message to the virtual environment 155.

The virtual environment 155 may process the response message, and based on the processing may determine whether the electrical signals output by the neurons 135 correspond to a target set by the virtual environment 155 (or other logic). The target may be unknown to the MEA interface 150 and/or MEA 105. If the electrical signals corresponded to the target, then the virtual environment 155 may use an API of the MEA interface 150 to send a positive reinforcement training signal to the MEA interface 150. The positive reinforcement training signal indicates that electrical signals output by the neurons 135 in response to the digital input signal satisfied some criterion of the virtual environment 155 (e.g., indicates that some target objective of the virtual environment was satisfied by the representation of the one or more electrical signals). Alternatively, in some embodiments no positive reinforcement training signal is generated or sent to the MEA interface. Also, if the electrical signals fail to correspond to the target, then the virtual environment 155 may use the API of the MEA interface 150 to send a negative reinforcement training signal to the MEA interface 150. The negative reinforcement training signal indicates that electrical signals output by the neurons 135 in response to the digital input signal failed to satisfy some criterion of the virtual environment 155 (e.g., indicates that some target objective of the virtual environment was not satisfied by the representation of the one or more electrical signals). Alternatively, in some embodiments no negative reinforcement training signal is generated or sent. Instead, all inputs to the neurons 135 may be paused for a brief time period if the electrical signals fail to correspond to the target. In one embodiment, positive reinforcement signals are used, but negative reinforcement signals are not used. In one embodiment, both positive and negative reinforcement signals are used. In one embodiment, negative reinforcement signals but not positive reinforcement signals are used.

In one embodiment, positive reinforcement signals are or include predictable signals, while negative reinforcement signals are or include unpredictable signals. A predictable signal may be a signal that follows a set pattern. Neurons (and the brain) are prediction machines, and seek to accurately predict external states either by making accurate predictions or by modifying the environment to make those predictions accurate. An implication of this suggests that unpredictable stimuli may be used as a form of punishment, and predictable stimuli may be used as a form of reward, whether the predictable or unpredictable stimuli is electrical stimuli, optical stimuli, or chemical stimuli. Predictable and unpredictable stimuli may be used in embodiments to shape the behavior of neurons. In an example, an MEA may include multiple sensory electrodes (also referred to as stimulus electrodes), such as 2-20 (e.g., 8 or 10) sensory electrodes or a continuum of sensory areas over a given predefined area. These sensory electrodes may deliver electrical signals according to one or more rules of a virtual environment 155 and/or training logic. Similarly, optical components (e.g., light sources) may deliver optical signals according to the one or more rules of the virtual environment 155 and/or training logic. This can train the neurons to expect these sensory electrodes (or some subset of the sensory electrodes) to receive electrical stimulation under certain predictable circumstances according to the rules of the virtual environment 155. Similarly, this can train the neurons to expect optical simulation under certain predictable circumstances according to the rules of the virtual environment. When such electrical and/or optical signals are received as expected, this acts as a reward to the neurons. However, electrical and/or optical signals may be delivered in a random manner or according to some other rule or rules that have not been applied for the virtual environment 155, which are all unpredictable electrical stimuli. For example, if the virtual environment is the game Pong, then one or more of the sensory electrodes that are associated with a location in proximity with a moving ball may be excited when the neurons cause a paddle to be moved in front of the moving ball, where the excitation of these sensory electrodes would be a predicable stimulus. However, if the paddle in the virtual environment 155 is not moved in front of the ball, then all or a random sampling of the sensory electrodes may be excited, where the excitation of these sensory electrodes would be an unpredictable stimulus. An unpredictable stimulus may be, for example, a random sequence of electrical and/or optical signals by a random selection of sensory electrodes, where the random sequence does not have any structure. Experimentation has shown that unpredictable stimuli may disrupt the internal dynamics of a biological neural network, and that predictable stimuli reinforces existing connections between neurons.

In some embodiments, neurons 135 are trained without using any positive or negative stimulus. There may be a steady or periodic stream of signals representative of or associated with the virtual environment 155 to neurons 135 during standard operation. Each set of signals may include analog signals delivered to appropriate electrodes 130 (or light emitting elements) to apply the optical or electrical impulses at specified coordinates and/or with specified intensity/amplitude, frequency/wavelength, and so on. For each set of signals, the neurons 135 may generate responses (e.g., by generating electrical impulses/signals). If the electrical signals generated by the neurons 135 corresponds to a target (e.g., is within a target range), then the stream of signals associated with the virtual environment may continue. However, if the electrical signals generated by the neurons 135 does not correspond to the target, then the stream of signals associated with the virtual environment may be paused for a period (e.g., 1-5 seconds), thus depriving the neurons 135 of any stimulus. Experimentation has shown that neurons effectively desire a stimulus, and will operate in a manner to increase the chance of receiving a stimulus. Accordingly, neurons can be trained to perform tasks by depriving the neurons of stimuli when they fail to act as desired. This is a different paradigm of learning from reinforcement learning, because in these embodiments there may be no explicitly set reward signals or punishment signals.

Responsive to receiving the training signal (which may be a reward signal or a punishment signal), MEA interface 150 may determine an electrical or chemical reward stimulus or punishment stimulus for the biological neurons 135 and/or may send an instruction to the MEA 105 to output the electrical or chemical reward or punishment stimulus. Alternatively, the MEA interface 150 may determine whether to continue providing stimuli associated with the virtual environment or to stop providing stimuli associated with the virtual environment for a time. The integrated circuit 145 may receive the instruction to output the electrical or chemical reward or punishment stimulus (or to continue or stop providing stimuli associated with the virtual environment), and may then cause the electrical or chemical reward or punishment stimulus to be output to the biological neurons 135 (or may permit stimuli associated with the virtual environment to continue or stop stimuli associated with the virtual environment from being delivered to the neurons 135).

In one embodiment, the reward or punishment stimulus is an electrical stimulus that may be delivered via the array of electrodes 130. For example, a reward stimulus may be an electrical impulse having a delta waveform. The electrical impulse having the delta waveform may be applied at multiple electrodes (e.g., at all of the electrodes in some embodiments) to deliver the electrical impulse to multiple locations in the array (e.g., in the 2D or 3D grid) to provide a deltoid stimulation to the biological neurons 135.

In one embodiment, a reward stimulus is a chemical reward stimulus. The MEA 105 may further include or be connected to one or more light sources that can emit light of a particular wavelength. These light sources can be activated by the integrated circuit 145 in embodiments. Additionally, the recording chamber 140 may include a protein disposed therein that is sensitive to the particular wavelength of light. The protein (e.g., an opsin protein) may be bound with dopamine or another compound or substance. When the protein is exposed to the particular wavelength of light, the protein may release some amount of the bound dopamine or other compound or substance.

In one embodiment, the reward stimulus includes tetanic stimulation of one or more neurons. A tetanic stimulation includes a high-frequency sequence of individual stimulations of a neuron (or group of neurons). In one embodiment, the high-frequency stimulation comprises a sequence of individual stimulations of one or more neurons delivered at a frequency of about 100 Hz or above. High-frequency stimulation causes an increase in the probability of a cell firing in the future called post-tetanic potentiation. The presynaptic event is caused by calcium influx on the presynaptic terminal, which ends up increasing the readily releasable pool of vesicles along with altering the release probability. Complex calcium-protein interactions then produce a change in vesicle exocytosis. The result of such changes causes the postsynaptic neuron to be more likely to fire an action potential when related pathways encounter voltage changes.

The chemical reward stimulus, electrical reward stimulus and tetanic stimulation form of reward stimulus all provide a form of reinforcement learning for the biological neurons 135. The punishment stimulus may also provide a form of reinforcement learning. The biological neurons 135 are rewarded when they generate electrical signals that satisfy some criteria of the virtual environment 155 and/or punished when they generate electrical signals that fail to satisfy the criteria, and over time will learn what the targets are and learn how to achieve those targets. The biological neurons 135 may be self-organizing, and may form connections to achieve the targets. In one embodiment, with each success of the biological neurons 135, the chemical or electrical reward stimulus is reduced (e.g., the amount of dopamine released is reduced). In one embodiment, the neurons 135 may learn via Hebbian learning. For example, if two neurons fire together to make something happen and are rewarded, then the next time it takes less activation or voltage to get those two neurons to fire again, thus increasing the frequency of this happening.

The digital input signal, instructions for electrical or optical impulses, representation of electrical and/or optical signals and/or response messages may be stored in a data store (e.g., for study and/or analysis). Researchers from around the world may access the stored data and/or the virtual environment for study via client computing device 125 connected to the network 120. For example, a researcher may develop an artificial or virtual environment 155 (e.g., a game), run an experiment that applies the game to the neurons 135, and receive data from the experiment. The data from the experiment may then be available to the researcher and/or other researchers via the cloud.

In one embodiment, the server computing device 110 further includes an artificial neural network (e.g., that may be external to virtual environment 155). The artificial neural network may be trained in parallel with the biological neural network comprising the neurons 135. For example, the digital input signal may be input into the artificial neural network, and a target associated with the digital input signal may be provided to the artificial neural network. The artificial neural network may be trained (e.g., using back propagation) at the same time that the biological neural network is trained.

In one embodiment, server computing devices(s) 110 include a compound analyzer 175 configured to perform operations regarding the testing of chemical compounds on neurons 135. Additionally, a chemical source 160 may deliver one or more chemical compounds such as pharmaceuticals to the neurons 135 via a chemical input 165. The chemical input 165 may be, for example, a tube that connects to the chemical source 160. A valve may be opened to deliver a precise dose of a chemical compound or mixture of chemical compounds to the neurons 135.

In one embodiment, once neurons 135 are trained to perform a task by virtual environment 155, a score or value may be determined that indicates a level of skill or degree of success of the neurons 135 at performing the task. There are many different types of tasks that the neurons 135 may be trained to perform. One example provided herein is the task of playing the computer game Pong. Pong is a simple “tennis-like” game that features two paddles and a ball. The goal of pong is to defeat an opponent (e.g., which may be a computer opponent provided by the virtual environment, an actual human opponent, or another set of trained neurons) by being the first one to gain 10 points. In Pong, a player receives a point once the opponent misses the ball (which occurs when they fail to move their paddle in front of the ball and allow the ball to move past their paddle to the edge of the screen). The neurons may be trained to perceive the Pong game area, including the moving ball and the two paddles, and to move one of the paddles to intercept the ball. A cognitive function value may be determined by compound analyzer 175 based on how well the trained neurons play the Pong game. For example, a cognitive function value may be based on a win to loss ratio of the neurons, based on how long the neurons are able to keep the ball in play, based on how many points the neurons can achieve before losing the Pong game, and so on.

In one embodiment, compound analyzer 175 determines a baseline cognitive function value for neurons 135. The baseline cognitive function value may be determined based on one or more attempts of the neurons to perform the task that they were trained to perform. In one embodiment, a cognitive function value is determined for each attempt of the neurons 135 to perform the task, and an average baseline cognitive function value is determined based on an average (e.g., a moving average) of the cognitive function values.

In one embodiment, compound analyzer 175 determines a baseline neuro criticality value for the neurons 135, as described above. The baseline neuro criticality value may be at or near a target criticality value, and may thus be considered to be at criticality (e.g., at a phase transition). In one embodiment, the baseline cognitive function value is or is based at least in part on the baseline neuro criticality value. In one embodiment, the baseline cognitive function value is distinct from the baseline criticality value. In such embodiments, the baseline cognitive function value and the baseline criticality value may be used together to establish a baseline cognitive function of the neurons 135.

Compound analyzer 175 may cause chemical source 160 to deliver a dose of one or more chemical compound via chemical input 165 and to expose the neurons 135 to the dose of the chemical compound. The neurons 135 may then be used to perform the task for which they were trained, and a post-exposure cognitive function value may be determined based on an ability of the neurons to perform the task while under an influence of the chemical compound. This process may be repeated multiple times to generate multiple post-exposure cognitive function values. An average post-exposure cognitive function value may be determined based on the multiple post-exposure cognitive function values. In some embodiments, the neurons 135 are exposed to different doses of the chemical compound at different times, and post-exposure cognitive function values (e.g., average post-exposure cognitive function values) are determined for each of the doses.

Compound analyzer 175 may compare one or more post-exposure cognitive function values to the baseline cognitive function values. Based on the comparison, compound analyzer 175 may determine whether the cognitive function values increase, decrease, or stay the same before and after exposure of the neurons to the chemical compound(s). If the post-exposure cognitive function value(s) are higher than the baseline cognitive function values, this is an indicator that cognitive function of the neurons increased as a result of exposure to the chemical compound. Additionally, an amount of improvement in the cognitive function can be quantified based on a magnitude of the difference in the cognitive function values. Similarly, if the post-exposure cognitive function value(s) are lower than the baseline cognitive function values, this is an indicator that cognitive function of the neurons decreased as a result of exposure to the chemical compound. Additionally, an amount of impairment in the cognitive function can be quantified based on a magnitude of the difference in the cognitive function values.

In one embodiment, compound analyzer 175 determines a post-exposure neuro criticality value for the neurons 135, as described above. The post-exposure neuro criticality value may be at, above, or below a target criticality value. If the post-exposure neuro criticality value is above the target criticality value, then the post-exposure neuro criticality value may be considered to be above criticality. If the post-exposure neuro criticality value is below the target criticality value, then the post-exposure neuro criticality value may be considered to be below criticality. In some embodiments, the post-exposure neuro criticality value may be compared to the baseline neuro criticality value, and any differences therebetween may be measured. Differences between the post-exposure criticality value and the baseline criticality value may be indicative of a change in the cognitive function of the neurons. For example, if the post-exposure criticality value is below the baseline criticality value, then the neurons may be considered to be cognitively impaired.

In one embodiment, the post-exposure cognitive function value is or is based at least in part on the post-exposure neuro criticality value. Alternatively, the post exposure cognitive function value may be based on other measures such as those outlined above, such as measures of information content, complexity, entropy, or a combination thereof. In some embodiments, the post-exposure cognitive function value is based on a combination of criticality and one or more of these other measures. In one embodiment, the post-exposure cognitive function value is distinct from the post-exposure criticality value. In such embodiments, the post-exposure cognitive function value and the post-exposure criticality value may be used together to establish a post-exposure cognitive function of the neurons 135.

Additionally, compound analyzer 175 may compare post-exposure cognitive function values and/or neuro criticality values associated with different doses of the chemical compound (or different combinations of doses of one or more chemical compounds) to one another and/or to the baseline cognitive function value(s) and/or neuro criticality value(s). Based on such comparison, compound analyzer 175 may determine whether cognitive function improvement and/or impairment increases or decreases with increased dose, and/or whether there is a peak dose beyond which no further improvement or impairment is measured. Many other insights can also be gained based on statistical analysis of the cognitive function values and/or neuro criticality values determined for the neurons 135 under exposure to one or more doses of the chemical compound. It should be understood that discussions herein below that relate to cognitive function values also apply to neuro criticality values, and that in embodiments the cognitive function values may be at least in part based on neuro criticality values.

FIG. 1B illustrates an example in vitro chemical compound testing system 170, in accordance with one embodiment. In vitro chemical compound testing system 170 may include some or all of the same components of biological computing platform 100 of FIG. 1A. In some embodiments, in vitro chemical compound testing system 170 corresponds to biological computing platform 100 of FIG. 1A. In vitro chemical compound testing system 170 includes server computing device(s) 110 having an MEA interface 150, one or more virtual environments 155 and compound analyzer 175. In vitro chemical compound testing system 170 may further include client compound devices 125, network 120, chemical source 160, and multiple MEAs, including MEA 105A, MEA 105B, through MEA 105N. Each of the MEAs 105A-N may include a respective sample or culture of neurons 135A, 135B, through 135N.

As described above with reference to FIG. 1A, each of the neurons 135A-N may be trained to perform a task. In embodiments, each of the neurons 135A-N is taught to perform the same task. Alternatively, different neurons may be trained to perform different tasks. Once neurons 135A-N are trained to perform a task, baseline cognitive function values are determined for each of the neurons 135A-N by compound analyzer 175. Subsequently, chemical source 160 may deliver one or more doses of one or more chemical compounds to the neurons 135A-N via chemical inputs 165. Post-exposure cognitive function values may then be determined for each of the neurons 135A-N by compound analyzer 175.

Compound analyzer 175 may then perform statistical analysis on the cognitive function values determined for the multiple sets of neurons 135A-N. In embodiments, different sets of neurons 135A-N may be from the same genetic stock (e.g., same cell line) or from different genetic stock. For example, multiple sets of neurons may be trained for each of a plurality of genotypes and/or for stem cell lines having different pathologies, and compound analyzer 175 may determine whether different effects are exhibited by different genotypes and/or neurons having particular pathologies when exposed to the chemical compound(s). Such information may be accessed by a pharmaceutical company via client computing devices 125, for example, which may use such data in assessing the benefits and/or risks of drugs under development.

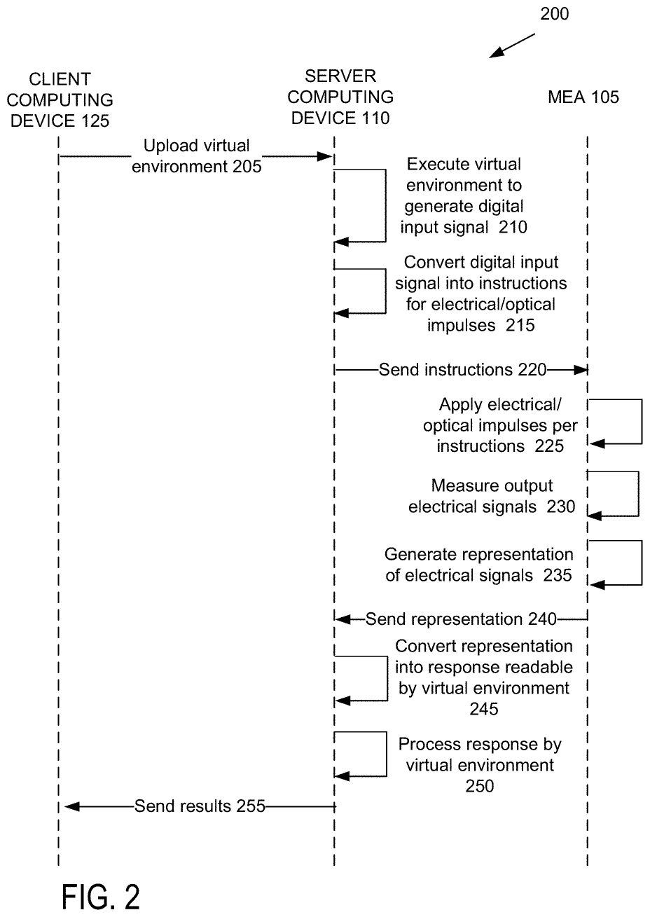

FIGS. 2-4 are flow diagrams and sequence diagrams illustrating methods of providing a biological computing platform, which may be used to assess the effect of chemical compounds on the cognitive function of neurons. These methods may be performed by processing logic of a server computing device as well as processing logic of an MEA, each of which may include hardware (e.g., circuitry, dedicated logic, programmable logic, microcode, etc.), software (such as instructions run on a processing device, a general purpose computer system, or a dedicated machine), firmware, or a combination thereof. The methods may be performed by an MEA and/or a server computing device. For example, some operations may be performed by an MEA and other operations may be performed by a computing device.

For simplicity of explanation, the methods are depicted and described as a series of acts. However, acts in accordance with this disclosure can occur in various orders and/or concurrently and with other acts not presented and described herein. Furthermore, not all illustrated acts may be performed to implement the methods in accordance with the disclosed subject matter. In addition, those skilled in the art will understand and appreciate that the methods could alternatively be represented as a series of interrelated states via a state diagram or events.

FIG. 2 is a sequence diagram illustrating one embodiment for a method 200 of using a biological computing platform. A client computing device 125 may upload 205 a virtual environment (also referred to as an artificial environment or experiment logic) to a server computing device 110. The server computing device 110 may then execute the virtual environment at block 210, and the virtual environment may generate a digital input signal. An MEA interface executing on the server computing device 110 may receive the digital input signal and convert it into instructions for electrical or optical impulses at block 215. The server computing device 110 may then send 220 the instructions to an MEA 105. In some embodiments, the digital input signal is readable by an integrated circuit (e.g., a processing device) of the MEA 105, and no conversion is performed at block 215. In such embodiments, the digital input signal may be sent to the MEA 105 and processed by the MEA at block 220.

The MEA 105 may generate one or more analog optical or electrical impulses (input signals) based on the instructions (or based on the digital input signal). The signals may be applied at specific electrodes that have specific locations (e.g., x,y coordinates or x,y,z coordinates) at block 225. At block 230, the MEA 105 may then measure output electrical signals generated by biological neurons of a biological neural network in the MEA 105. The MEA (or optical component) may then generate a representation of the electrical signals at block 235. This may include using an analog to digital converter to convert the analog electrical signals into digital values.

At block 240, the MEA sends the representation of the measured electrical signals output by the neurons to the server computing device. At block 245, the MEA interface on the server computing device may convert the representation into a response message for the virtual environment that is readable by processing logic of the virtual environment. The MEA interface may then send the response message to the virtual environment. At block 250, the virtual environment may then process the response message. In some embodiments, the representation of the electrical signals is readable by the virtual environment, and no conversion is performed at block 245. In such embodiments, the representation may be sent to the virtual environment and processed by the virtual environment. The virtual environment may then generate results, which the server computing device 110 may send to the client computing device 125.

In embodiments, the blocks 210-250 form a loop that is continuously run until some stop signal is applied. For example, after the operations of block 250 are completed, the method may return to block 210, and the operations of block 210 may be repeated.

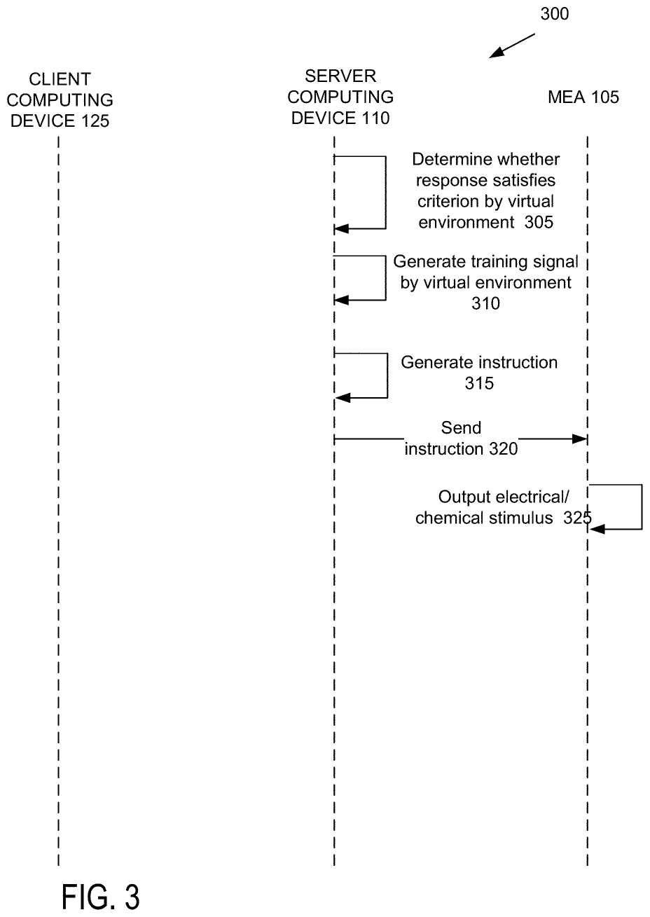

FIG. 3 is a sequence diagram illustrating one embodiment for a method 300 of providing reinforcement learning to biological neurons in a biological computing platform. Method 300 may be performed after method 200 is completed. At block 305, the virtual environment executing on the server computing device 110 determines whether a response message (or representation of electrical signals output by neurons on the MEA 105) satisfies some criterion. If the response message (or representation of electrical signals) satisfies the criterion, then the virtual environment may generate a first training signal and provide the first training signal to the MEA interface executing on the server computing device 110. If the response message (or representation of electrical signals) fails to satisfy the criterion, then the virtual environment may generate a second training signal and provide the second training signal to the MEA interface executing on the server computing device 110. The MEA interface may then generate a reward instruction based on the first training signal or a punishment instruction based on the second training signal (block 315) and send the reward or punishment instruction to MEA 105 (block 320). Alternatively, the MEA interface may forward the training signal to the MEA 105. At block 325, the MEA may then output an electrical or chemical reward stimulus or punishment stimulus to the biological neural network on the MEA 105, as appropriate.

In one example, the virtual environment includes a video game such as Pong. In such an embodiment, the digital input signal may be a projection of the game world (e.g., a frame of a display or user interface of the game). In one embodiment, the projection of the game world is a mapping of the pixels of the display for the game at a given point in time. Each pixel in the display may be associated with a location in a 2D grid of electrodes on the MEA 105. Depending on the resolution of the display and a number of rows and columns in the 2D grid of electrodes, there may be a 1 to 1 mapping between pixels of the display and electrodes in the MEA, a 1 to X mapping or an X to 1 mapping, where X is a positive integer. An MEA interface running on the server computing device 110 may determine a mapping between the pixels of the display for the game and the 2D grid in the MEA 105. For example, x,y pixel 1,3 may map to an electrode at column 2, row 6 of the 2D grid. In one embodiment, converting the digital input signal at block 215 includes determining, for each location of the 2D grid, whether the location is to be activated (with an impulse sent to the electrode at the location) or deactivated (with no impulse sent to the electrode at the location). Accordingly, the electrical or optical signals may be applied at the specified activated locations. In the example of Pong, the instructions for electrical/optical impulses may represent a court, a position of a ball and positions of paddles in the court.

In another example mapping system, features such as position of ball in the court space (x,y), position of paddle as (z), distance from (d) of ball to the paddle, etc. are mapped as discrete values where regions are arbitrarily assigned and stimulated on the surface of the chip.

In the above examples the biological neural network may be trained to move the paddle to intercept the ball. This may be achieved by demarcating the 2D grid in the MEA 105 into 4 quadrants. With each set of electrical/optical impulses that are applied to the biological neural network, the electrical signals generated by the neurons may be measured. If a majority of electrical signals measured are from an upper right quadrant, then this may cause the virtual environment to move the right paddle up. If a majority of electrical signals measured are from a lower right quadrant, then this may cause the virtual environment to move the right paddle down. A positive reward stimulus may then be provided to the biological neural network when the ball intercepts the right paddle, as discussed above.

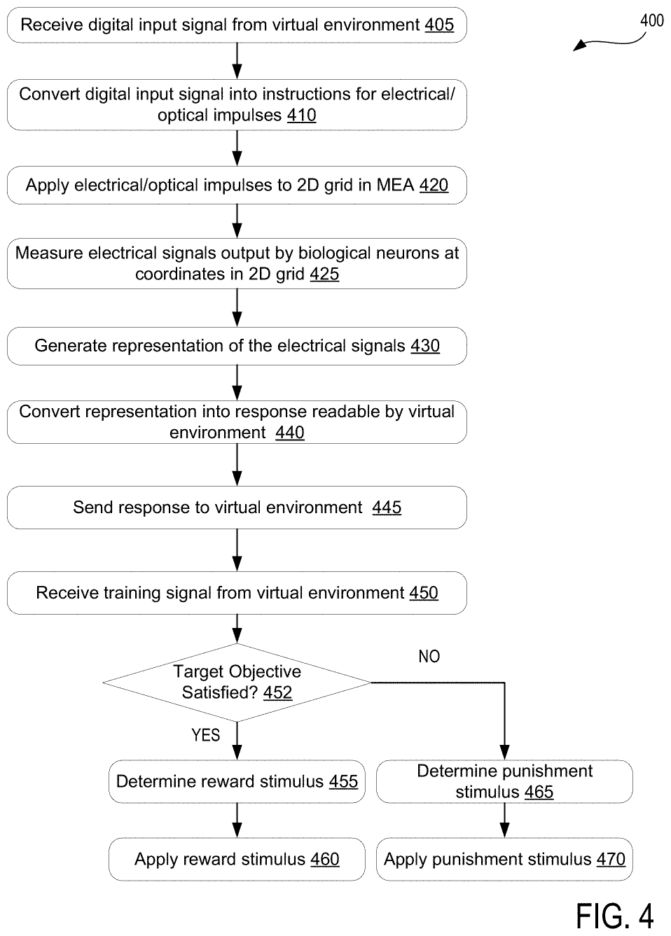

FIG. 4 is a flow diagram illustrating one embodiment for a method 400 of using a biological computing platform. At block 405, a computing device receives a digital input signal from a virtual environment. At block 410, the computing device converts the digital input signal into instructions for electrical/optical impulses. At block 420, the computing device provides the instructions to an MEA, which applies the electrical/optical impulses to an array of electrodes (e.g., a 2D grid or 3D matrix of electrodes).

At block 425, the MEA or other component measures electrical signals output by biological neurons at coordinates of the array (e.g., at coordinates of the 2D grid or 3D matrix). The electrical signals may be analog signals. At block 430, the MEA may generate a digital representation of the electrical signals. At block 440, the MEA may send the digital representation to the computing device, which may convert the digital representation into a response message for the virtual environment. At block 445, the computing device may provide the response message to the virtual environment.

At block 450, the computing device may receive a training signal from the virtual environment. At block 452, processing logic may determine whether one or more criteria (e.g., a target objective) was satisfied and/or whether the training signal is a reward (positive reinforcement) signal or a punishment (negative reinforcement) signal. If the objective was satisfied and/or the training signal was a reward signal, the method continues to block 455. If the objective was not satisfied and/or the training signal was a punishment signal, the method continues to block 465.

At block 455, the computing device may determine an electrical or chemical reward stimulus (e.g., a predictable stimulus) and instruct the MEA to apply the electrical or reward stimulus. Alternatively, the computing device may forward the training signal to the MEA. At block 460, the MEA then applies the electrical or reward stimulus to the biological neurons.

At block 465, the computing device may determine an electrical or chemical punishment stimulus (e.g., an unpredictable stimulus) and instruct the MEA to apply the electrical or punishment stimulus. Alternatively, the computing device may forward the training signal to the MEA. At block 470, the MEA then applies the electrical or chemical punishment stimulus to the biological neurons.

This process may continue, and in turn a biological neural network may be trained.

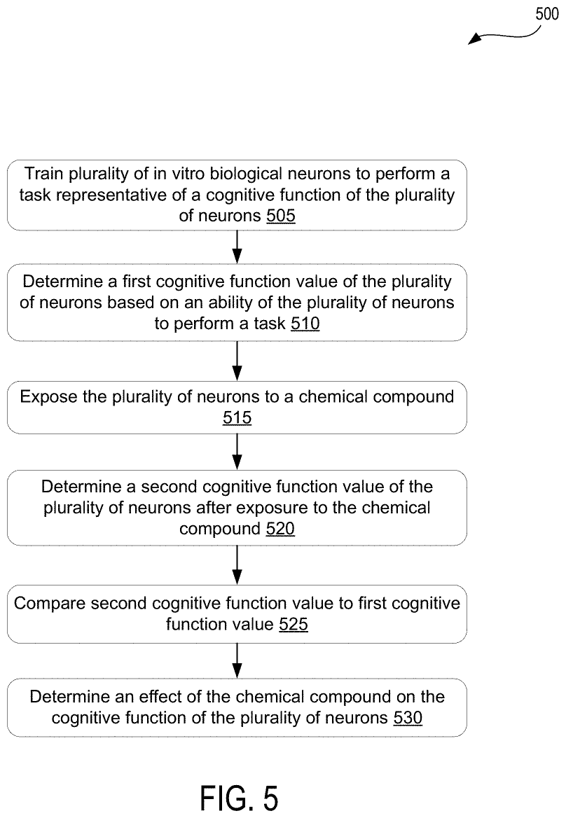

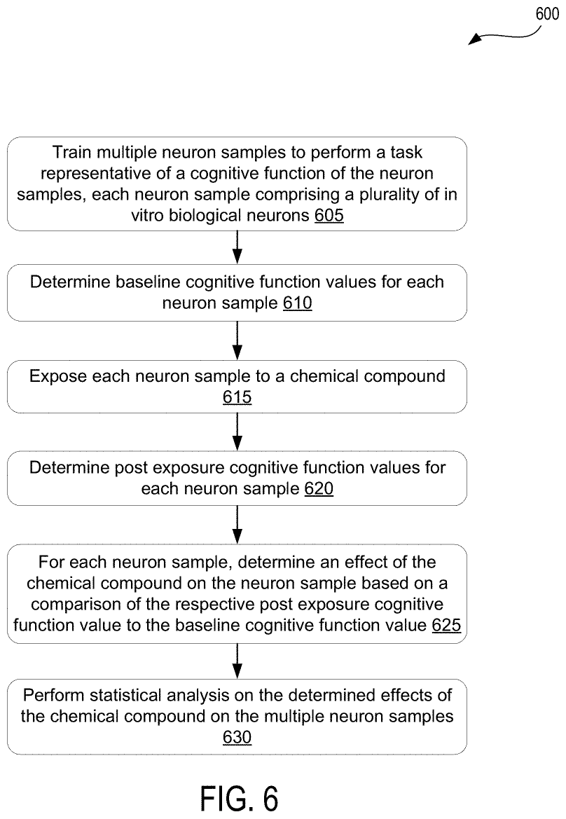

FIGS. 5-6 are flow diagrams illustrating methods of performing in vitro testing of the effects of one or more chemical compounds on the cognitive function of neurons. These methods may be performed by processing logic of a computing device (e.g., a server computing device) as well as processing logic of an MEA, each of which may include hardware (e.g., circuitry, dedicated logic, programmable logic, microcode, etc.), software (such as instructions run on a processing device, a general purpose computer system, or a dedicated machine), firmware, or a combination thereof. The methods may be performed by an MEA and/or a computing device. For example, some operations may be performed by an MEA and other operations may be performed by a computing device.

For simplicity of explanation, the methods are depicted and described as a series of acts. However, acts in accordance with this disclosure can occur in various orders and/or concurrently and with other acts not presented and described herein. Furthermore, not all illustrated acts may be performed to implement the methods in accordance with the disclosed subject matter. In addition, those skilled in the art will understand and appreciate that the methods could alternatively be represented as a series of interrelated states via a state diagram or events.

Referring to FIG. 5, at block 505 of method 500 processing logic trains a plurality of in vitro biological neurons to perform a task. The degree of success of the neurons to perform the task may be representative of a cognitive function of the plurality of neurons. The neurons may be trained to perform the task as discussed herein above. At block 510, processing logic determines a first cognitive function value of the plurality of neurons based on an ability of the neurons to perform the task for which they were trained. The first cognitive function value may be a measure of how well the task was performed, how long it took to complete the task, how long the neurons were able to continue performing the task, and so on. In an example, the task may include multiple milestones and/or be divided into subtasks, and the first cognitive function value may be based on the number of milestones that were achieved and/or the number of subtasks that were completed. The first cognitive function value may be a baseline cognitive function value.

At block 515, the plurality of neurons are exposed to a chemical compound. The neurons may be exposed to a particular dosage of the chemical compound. Alternatively, the neurons may be exposed to a mixture of multiple chemical compounds. The mixture may include a determined dosage for each of the chemical compounds in the mixture, where the same or different dosages may be used for different chemical compounds. In one embodiment, the chemical compound (or at least one of the multiple chemical compounds) is a drug of interest.

At block 520, processing logic determines a second cognitive function value of the plurality of neurons during and/or after exposure of the plurality of neurons to the chemical compound (or mixture of chemical compounds). At block 525, processing logic compares the second cognitive function value to the first cognitive function value. At block 530, processing logic determines an effect of the chemical compound(s) on the cognitive function of the plurality of neurons based on a result of the comparison.

Method 500 may be repeated multiple times for the same set of neurons under the same or different conditions (e.g., under different exposure conditions of the neurons to the chemical compound). Additionally, or alternatively, method 500 may be performed for many different sets of neurons (e.g., each in a different MEA). The different sets of neurons may be from the same genetic stock or from different genetic stock (e.g., having different genotypes and/or pathologies). The results of the multiple tests may be analyzed using statistical analysis, as discussed with reference to FIG. 6.

Referring now to FIG. 6, at block 605 of method 600 processing logic trains multiple neuron samples (each containing a plurality of in vitro biological neurons) to perform a task representative of a cognitive function of the neuron samples. In some embodiments, all neuron samples are trained to perform the same task. Alternatively, different neuron samples may be trained to perform different tasks. For example, a first set of neuron samples (each in its own MEA) may be trained to perform a first task and a second set of neuron samples (each in its own MEA) may be trained to perform a second task.

At block 610, processing logic determines baseline cognitive function values for each of the trained neuron samples. At block 615, processing logic exposes each neuron sample to a chemical component (or mixture of chemical components). Different neuron samples may be exposed to the same or different doses of the chemical compound. Additionally, different tests may be performed on the same neuron sample, where for each test the same or a different dosage of the chemical compound may be used.

At block 620, processing logic determines post exposure cognitive function values for each of the neuron samples, and for each of the tests performed on those neuron samples. For example, multiple post exposure cognitive function values may be determined for the same neuron sample over a time period to obtain a statistically significant amount of data points for that neuron sample. This may include continuing to determine post exposure cognitive function values after the neuron sample has been exposed to the chemical compound, each at a different time offset from when the neuron sample was exposed to the chemical compound. Additionally, or alternatively, multiple different dosages of the chemical compound may be tested on the same neuron sample or samples, and one or more post exposure cognitive function values may be determined for each dosage amount.

At block 625, processing logic determines, for each neuron sample, an effect of the chemical compound on the neural sample based on a comparison of the respective post exposure cognitive function value(s) to the baseline cognitive function value(s) for that neuron sample. This may include determining the effect of the chemical compound on the cognitive function of the neuron sample over time by comparing multiple post exposure cognitive function values, each associated with a different length of time from when the neuron sample was exposed to the chemical compound. Such information may be used to determine how long the chemical compound affects the cognitive function of the neuron sample, how fast the effects of the chemical compound on the cognitive function of the neuron sample wear off, and/or whether a diminishment of the effects of the chemical compound on the neuron sample over time is linear or non-linear. For example, a shape of a curve defining the effects of the chemical compound on the cognitive function of the neuron sample over time may be determined.

At block 630, processing logic performs statistical analysis on the determined effects of the chemical compound on the multiple neuron samples. Such data may be used to determine the efficacy of a drug as it applies to cognitive function, side effects of a drug as it applies to cognitive function, and so on. Such statistical analysis may also be used to determine whether the effects on cognitive function vary by genotype, by pathology and/or by other variables, for example. Such analysis may also be performed to test therapeutic windows to determine minimum efficacy levels and/or toxicity limits of the chemical compound. Additionally, patient specific lines may be used to test different genetic backgrounds.

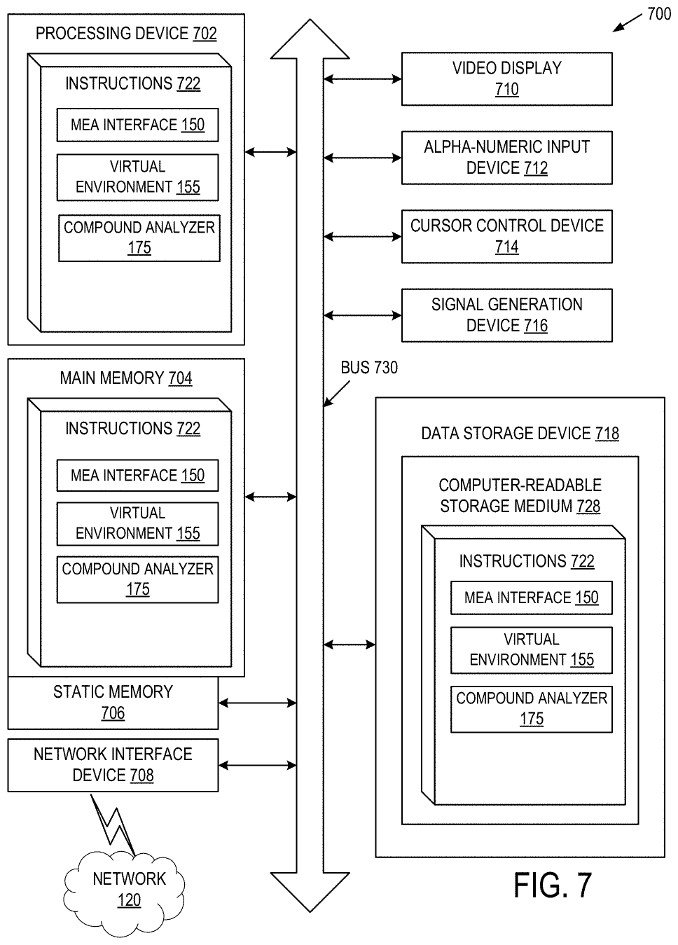

FIG. 7 illustrates a diagrammatic representation of a machine in the example form of a computing device 700 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 700 includes a processing device 702, a main memory 704 (e.g., read-only memory (ROM), flash memory, dynamic random access memory (DRAM) such as synchronous DRAM (SDRAM) or Rambus DRAM (RDRAM), etc.), a static memory 706 (e.g., flash memory, static random access memory (SRAM), etc.), and a secondary memory (e.g., a data storage device 718), which communicate with each other via a bus 730.

Processing device 702 represents one or more general-purpose processors such as a microprocessor, central processing unit, or the like. More particularly, the processing device 702 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. Processing device 702 is configured to execute the processing logic (instructions 722) for performing the operations and steps discussed herein.

The computing device 700 may further include a network interface device 708. The computing device 700 also may include a video display 710 (e.g., a liquid crystal display (LCD) or a cathode ray tube (CRT)), an alphanumeric input device 712 (e.g., a keyboard), a cursor control device 714 (e.g., a mouse), and/or a signal generation device 716 (e.g., a speaker).

The data storage device 718 may include a machine-readable storage medium (or more specifically a computer-readable storage medium) 728 on which is stored one or more sets of instructions 722 embodying any one or more of the methodologies or functions described herein. The instructions 722 may also reside, completely or at least partially, within the main memory 704 and/or within the processing device 702 during execution thereof by the computer system 700, the main memory 704 and the processing device 702 also constituting computer-readable storage media.

The computer-readable storage medium 728 may also be used to store MEA interface 150, compound analyzer 175 and/or virtual environment 155 (as described with reference to the preceding figures), and/or a software library containing methods that call MEA interface 150, compound analyzer 175 and/or virtual environment 155. While the computer-readable storage medium 728 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium that is capable of storing or encoding a set of instructions for execution by the machine and that cause the machine to perform any one or more of the methodologies described herein. The term “non-transitory computer-readable storage medium” shall accordingly be taken to include, but not be limited to, solid-state memories, and optical and magnetic media.

Some portions of the detailed description have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving”, “converting”, “sending”, or the like, may refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments of the present disclosure also relate to an apparatus for performing the operations herein. This apparatus may be specially constructed for the discussed purposes, and/or it may comprise a general purpose computer system selectively programmed by a computer program stored in the computer system. Such a computer program may be stored in a computer readable storage medium, such as, but not limited to, any type of disk including floppy disks, optical disks, CD-ROMs, and magnetic-optical disks, read-only memories (ROMs), random access memories (RAMs), erasable programmable read only memories (EPROMs), electrically erasable programmable read only memories (EEPROMs), magnetic disk storage media, optical storage media, flash memory devices, other type of machine-accessible storage media, or any type of media suitable for storing electronic instructions, each coupled to a computer system bus.

It is to be understood that the above description is intended to be illustrative, and not restrictive. Many other embodiments will be apparent to those of skill in the art upon reading and understanding the above description. Although the present disclosure has been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method of performing in vitro testing of cognitive function, comprising:
training a first plurality of in vitro biological neurons to perform a task, wherein an ability of the first plurality of biological neurons to perform the task is representative of a cognitive function of the first plurality of in vitro biological neurons;
determining a first cognitive function value of the first plurality of in vitro biological neurons based on an ability of the first plurality of in vitro biological neurons to perform the task;
exposing the first plurality of in vitro biological neurons to a chemical compound;
determining a second cognitive function value of the first plurality of in vitro biological neurons based on the ability of the first plurality of in vitro biological neurons to perform the task during or after exposure to the chemical compound;
comparing the second cognitive function value to the first cognitive function value; and
determining an effect of the chemical compound on the cognitive function of the first plurality of in vitro biological neurons based on a difference between the second cognitive function value and the first cognitive function value.

2. The method of claim 1, wherein the first plurality of in vitro biological neurons are disposed on a multielectrode array (MEA) or a substrate comprising an optical system.

3. The method of claim 2, wherein training the first plurality of in vitro biological neurons disposed on the MEA or the substrate comprising the optical system to perform the task comprises:
receiving a digital input signal from a processing logic;
converting the digital input signal into instructions for a plurality of electrical or optical impulses, where each electrical or optical impulse of the plurality of electrical or optical impulses is associated with a two-dimensional (2D) coordinate;
applying the plurality of electrical or optical impulses at specified coordinates of a 2D grid in the MEA in accordance with the instructions;
measuring electrical or optical signals output by one or more of the first plurality of in vitro biological neurons at one or more additional coordinates of the 2D grid;
generating a representation of the electrical or optical signals;
sending a response to the processing logic based on the representation of the electrical or optical signals;
receiving a training signal from the processing logic, wherein the training signal indicates whether a target objective of the processing logic was satisfied by the representation of the electrical signals;
determining a stimulus for the first plurality of in vitro biological neurons based on whether the target objective of the processing logic was satisfied; and
outputting the stimulus to the first plurality of in vitro biological neurons.

4. The method of claim 3, wherein the training signal indicates that the target objective of the processing logic was satisfied, and wherein the stimulus is a chemical, optical or electrical reward stimulus.

5. The method of claim 4, wherein the stimulus comprises a predictable stimulus that follows a set pattern.

6. The method of claim 3, wherein the training signal indicates that the target objective of the processing logic was not satisfied, and wherein the stimulus is a chemical, optical or electrical punishment stimulus.

7. The method of claim 6, wherein the punishment stimulus comprises an unpredictable stimulus that fails to follow any set pattern.

8. The method of claim 7, wherein the unpredictable stimulus comprises electrical or optical impulses at a random selection of locations and/or delivered with random timing.

9. The method of claim 1, wherein the chemical compound comprises a drug.

10. The method of claim 1, wherein the first plurality of in vitro biological neurons comprise a first plurality of synthetic human neurons generated from a first stem cell source having a first genotype.

11. The method of claim 10, further comprising:
determining the effect of the chemical compound on the cognitive function of a second plurality of in vitro biological neurons generated from a second stem cell source having a second genotype that is different from the first genotype; and
comparing the effect of the chemical compound on the cognitive function of the second plurality of in vitro biological neurons to the effect of the chemical compound on the cognitive function of the first plurality of in vitro biological neurons; and
determining whether the effect of the chemical compound has a dependence on genotype based at least in part on the comparing.

12. The method of claim 1, wherein the first plurality of in vitro biological neurons comprises a plurality of animal neurons from an embryotic source.

13. The method of claim 1, further comprising:
determining that the effect of the chemical compound on the cognitive function is a deleterious effect that reduces the cognitive function of the of the first plurality of in vitro biological neurons.

14. The method of claim 1, further comprising:
determining that the effect of the chemical compound on the cognitive function is a beneficial effect that improves the cognitive function of the of the first plurality of in vitro biological neurons.

15. The method of claim 1, wherein the first plurality of in vitro biological neurons exhibit a known pathology.

16. The method of claim 1, wherein the first plurality of in vitro biological neurons are from a generic cell line.

17. The method of claim 1, wherein the first cognitive function value comprises a first neuro criticality value, and wherein the second cognitive function value comprises a second neuro criticality value.

18. An in vitro chemical compound testing system, comprising:
a first multielectrode array (MEA) comprising:
a two-dimensional (2D) grid of excitation sites;
a first plurality of in vitro biological neurons disposed on the MEA;
a processing device to apply a plurality of electrical or optical impulses at excitation sites having coordinates on the 2D grid of excitation sites; and
one or more sensors to measure signals output by one or more of the first plurality of in vitro biological neurons at coordinates of the 2D grid, wherein the processing device is to receive the signals from the one or more sensors and generate a representation of the signals;
a first chemical input configured to expose the first plurality of in vitro biological neurons to a chemical compound; and
a computing device connected to the MEA, wherein the computing device is to:
train the first plurality of in vitro biological neurons to perform a task, wherein an ability of the first plurality of biological neurons to perform the task is representative of a cognitive function of the first plurality of in vitro biological neurons;
determine a first cognitive function value of the first plurality of in vitro biological neurons based on an ability of the first plurality of in vitro biological neurons to perform the task before exposure to the chemical compound;
determine a second cognitive function value of the first plurality of in vitro biological neurons based on the ability of the first plurality of in vitro biological neurons to perform the task during or after exposure to the chemical compound;
compare the second cognitive function value to the first cognitive function value; and
determine an effect of the chemical compound on the cognitive function of the first plurality of in vitro biological neurons based on a difference between the second cognitive function value and the first cognitive function value.

19. The in vitro chemical compound testing system of claim 18, wherein to train the first plurality of in vitro biological neurons to perform the task, the computing device is to:
receive a digital input signal;
convert the digital input signal into instructions for the plurality of electrical or optical impulses;
send the instructions to the MEA;
receive the representation of the signals from the MEA;
process the representation of the signals;
receive a training signal, wherein the training signal indicates whether a target objective was satisfied by the representation of the electrical signals;
determine a stimulus for the first plurality of in vitro biological neurons based on whether the target objective was satisfied; and
send, to the MEA, an instruction to output the stimulus to the first plurality of in vitro biological neurons.

20. The in vitro chemical compound testing system of claim 18, wherein the first plurality of in vitro biological neurons comprise a first plurality of synthetic human neurons generated from a first stem cell source having a first genotype.

21. The in vitro chemical compound testing system of claim 20, further comprising:
a second MEA comprising a second plurality of in vitro biological neurons disposed on the second MEA, the second plurality of in vitro biological neurons having been generated from a second stem cell source having a second genotype that is different from the first genotype; and
a second chemical input configured to expose the second plurality of in vitro biological neurons to the chemical compound;
wherein the computing device is further to:
determine the effect of the chemical compound on the cognitive function of the second plurality of in vitro biological neurons;
make a comparison of the effect of the chemical compound on the cognitive function of the second plurality of in vitro biological neurons to the effect of the chemical compound on the cognitive function of the first plurality of in vitro biological neurons; and
determine whether the effect of the chemical compound has a dependence on genotype based at least in part on the comparison.
